(19) United States
(12) Patent Application Publication
(10) Pub. No.: US 2021/0251767 A1
(43) Pub. Date: Aug. 19, 2021

(54) Systems and Methods for Reverse Arthroplasty

(71) Applicant: Mayo Foundation for Medical Education and Research, Rochester, MN (US)

(72) Inventor: Cory Stewart, Eau Claire, WI (US)

(21) Appl. No.: 17/172,735

(22) Filed: Feb. 10, 2021

Related U.S. Application Data
(60) Provisional application No. 62/975,973, filed on Feb. 13, 2020.

(51) Int. Cl. A61F 2/40

(52) U.S. Cl. CPC A61F 2/4014; A61F 2002/30576; A61F 2002/4018; A61F 2002/4051

(57) ABSTRACT

Systems for arthroplasty, and methods of designing an arthroplasty system, such as in reverse arthroplasty, are provided that include an increased impingement-free range of motion. The systems and methods provide for a geometry of the reverse components to address the deficit of limited range of motion included with conventional reverse arthroplasty and thereby enhance a patient's quality of life.

### CROSS-REFERENCE TO RELATED APPLICATIONS

This application claims the benefit of U.S. Provisional Patent Application Ser. No. 62/975,973 filed on Feb. 13, 2020 and entitled “Systems and Methods for Reverse Arthroplasty,” which is incorporated herein by reference as if set forth in its entirety for all purposes.

### STATEMENT REGARDING FEDERALLY SPONSORED RESEARCH

Not Applicable.

### BACKGROUND

Two broad categories of shoulder replacements exist currently, anatomic or total shoulder arthroplasty replacements, and reverse shoulder replacements. Over 50,000 shoulder arthroplasty procedures are performed yearly in the United States with significantly more performed worldwide. Reverse arthroplasty replacement procedures are commonly performed. When comparing a reverse shoulder replacement to traditional anatomic shoulder replacements, there are significant restrictions relating to internal rotation of the shoulder with a reverse shoulder replacement, impacting a patient's quality of life and activities of daily living. There are, however, several scenarios where only a reverse shoulder arthroplasty can be performed. These include: patients who have had prior shoulder replacement surgeries, patients with significant changes in the shape or orientation of the bones comprising the shoulder joint, and patients with rotator cuff pathology, among others.

Reverse total shoulder arthroplasties should result in similar range of motion and function as anatomic shoulder replacements, except as it relates to internal rotation, or one's ability to bring one's hand behind the back. As such, existing technologies are not adequately meeting patient's needs as it relates to maximizing function after shoulder replacement surgery. Internal rotation behind the back is critical for several self-care tasks, and one's inability to bring the hand to the midline of the back presents significant limitations as it relates to activities of daily living. This limitation as it relates to internal rotation is related to several factors, but one factor is related to impingement of the replacement components in the front of the shoulder (anterior impingement).

Thus there remains a need for a reverse arthroplasty system that meets patient's needs as it relates to maximizing function after joint replacement surgery and provides for an increased range of motion (ROM) without failure. Such a system could provide for internal rotation of the shoulder, and enhance a patient's quality of life and activities of daily living.

### SUMMARY OF THE DISCLOSURE

The present disclosure addresses the aforementioned drawbacks by providing systems for reverse arthroplasty, and methods of designing a reverse arthroplasty system, that provide an increased impingement-free range of motion. The methods for design provide for a geometry of the reverse components to address the deficit of limited range of motion and thereby enhance a patient's quality of life. In a non-limiting example, a limitless reverse shoulder arthroplasty system is provided for a humeral component that includes increased range of impingement free motion for reverse shoulder replacements.

In one configuration, a joint prosthesis is provided. The joint prosthesis includes an implant dimensioned to be implanted in a first bone of a joint of a subject. The joint prosthesis also includes a prosthetic insert having an insert flat surface and an outer surface dimensioned for articulation with an articular surface of an artificial joint surface of a second bone of the joint. The prosthetic insert includes an extension opposite the outer surface of the insert dimensioned to be impacted into a well in a prosthetic baseplate. The joint prosthesis also includes the prosthetic baseplate with a baseplate flat surface configured to align with the insert flat surface. A location of the baseplate flat surface and insert flat surface is configured to provide a range of motion for the subject.

In some configurations, the joint prosthesis is a reverse prosthesis and the location of the baseplate flat surface and insert flat surface is an anterior location when the prosthetic insert and the prosthetic baseplate are implanted in the subject. In some configurations, the extension of the joint prosthesis is dimensioned to be impacted into the well in the prosthetic baseplate thereby forming an interference fit between the prosthetic insert and the prosthetic baseplate.

In some configurations, the joint prosthesis includes a mounting stud coupled to the prosthetic baseplate. The mounting stud may include a first end and a second end, the first end being coupled to the well, the second end being dimensioned for insertion into an opening in the implant dimensioned to be implanted in the first bone thereby forming an interference fit between the prosthetic baseplate and the implant dimensioned to be implanted in the first bone. In some configurations, the second end of the mounting stud includes an outer surface that tapers inward from the first end to an outermost section of the second end of the mounting stud.

In some configurations, the prosthetic baseplate includes locking tab extensions and the prosthetic insert includes provisions for receiving the locking tabs. The locking tab extensions may engage with the provisions thereby forming an interference fit between the prosthetic insert and the prosthetic baseplate. In some configurations, the joint prosthesis includes a plurality of locking tab extensions. The locking tab extensions may provide for alignment of the baseplate flat surface and insert flat surface.

In some configurations of the joint prosthesis, the first bone is a humerus and the second bone is a scapula.

In one configuration, a method is provided for manufacturing a prosthetic component for replacing a part of a bone of a joint in a subject. The method includes forming the prosthetic component to include a range of motion for the prosthetic component having been determined by: a) dimensioning an implant to be implanted in a first bone of a joint of a subject; b) forming a prosthetic insert having an insert flat surface and an outer surface dimensioned for articulation with an articular surface of an artificial joint surface of a second bone of the joint, the prosthetic insert including an extension opposite the outer surface of the insert dimensioned to be impacted into a well in a prosthetic baseplate; c) forming the prosthetic baseplate having a baseplate flat surface configured to align with the insert flat surface, and d) locating the baseplate flat surface and insert flat surface to provide a range of motion for the subject.

In some configurations of the method, the prosthetic component is a reverse prosthesis and the location of the baseplate flat surface and insert flat surface is an anterior location when the prosthetic insert and the prosthetic baseplate are implanted in the subject. In some configurations, the method includes impacting the extension into the well in the prosthetic baseplate thereby forming an interference fit between the prosthetic insert and the prosthetic baseplate.

In some configurations, the method includes coupling a mounting stud to the prosthetic baseplate, wherein the mounting stud includes a first end and a second end, the first end being coupled to the well, the second end being dimensioned for insertion into an opening in the implant dimensioned to be implanted in the first bone thereby forming an interference fit between the prosthetic baseplate and the implant dimensioned to be implanted in the first bone. An outer surface of the second end of the mounting stud may taper inwardly from the first end to an outermost section of the second end of the mounting stud.

In some configurations of the method, the prosthetic baseplate includes locking tab extensions and the prosthetic insert includes provisions for receiving the looking tabs. The method may include engaging the locking tab extensions with the provisions thereby forming an interference fit between the prosthetic insert and the prosthetic baseplate. In some configurations, the joint prosthesis includes a plurality of locking tab extensions. In some configurations, the method includes aligning the locking tab extensions of the prosthetic baseplate with the provisions of the prosthetic insert to align the baseplate flat surface with the insert flat surface.

In some configurations of the method, the first bone is a humerus and the second bone is a scapula.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a perspective view of a conventional reverse shoulder prosthesis.

FIG. 2 is an anterior view, partially in cross section, of one embodiment of a shoulder prosthesis in accordance with the present disclosure.

FIG. 3A is a top view of a non-limiting example bearing surface insert in accordance with the present disclosure.

FIG. 3B is a side view of the non-limiting example bearing surface insert of FIG. 3A in a direction of line 11-11 of FIG. 2.

FIG. 3C is a bottom perspective view of the non-limiting example bearing surface insert of FIG. 3A.

FIG. 4A is a top view of a non-limiting example tray baseplate in accordance with the present disclosure.

FIG. 4B is a side perspective view of the non-limiting example tray baseplate of FIG. 4A in accordance with the present disclosure.

FIG. 4C is a bottom perspective view of the non-limiting example tray baseplate of FIG. 4A in accordance with the present disclosure.

FIG. 5 is an exploded perspective view, partially in cross section, of a non-limiting example tray assembly.

FIG. 6 is a perspective view of an assembled non-limiting example tray assembly in accordance with the present disclosure.

### DETAILED DESCRIPTION

Systems for reverse arthroplasty are provided that include an increased impingement-free range of motion. Methods of designing, a reverse arthroplasty system that provides an increased impingement free range of motion are also provided. In a non-limiting example, a reverse shoulder arthroplasty system is provided for a humeral component that includes increased range of impingement free motion for reverse shoulder replacements. The reverse shoulder arthroplasty system may also increase the room for repair of the subscapularis. Subscapularis repair has been shown to minimize dislocation risk in several studies.

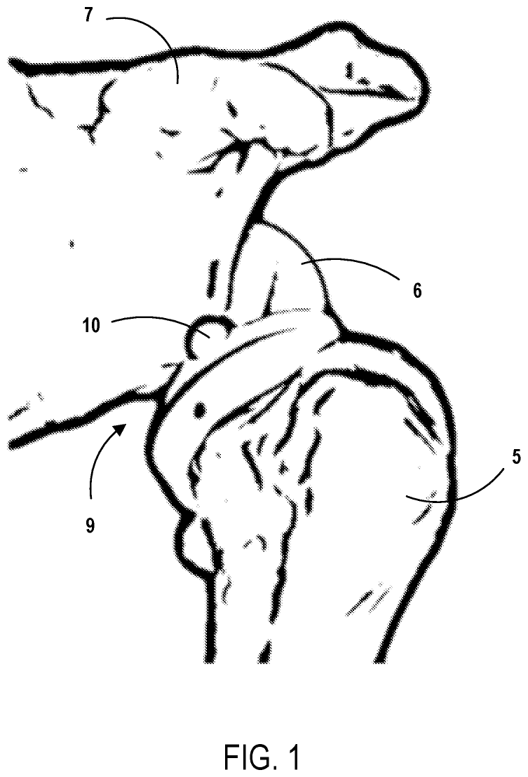

Referring to FIG. 1, an image is shown of a humerus 5 and a scapula 7, where the scapula 7 includes a glenoid component 6 and humerus 5 includes a conventional reverse implant 9. The conventional reverse implant 9 is round or circular when viewed from the face of the implant. Upon internal rotation, the circular conventional implant 9 experiences impingement at location 10, thus limiting range of motion for a subject.

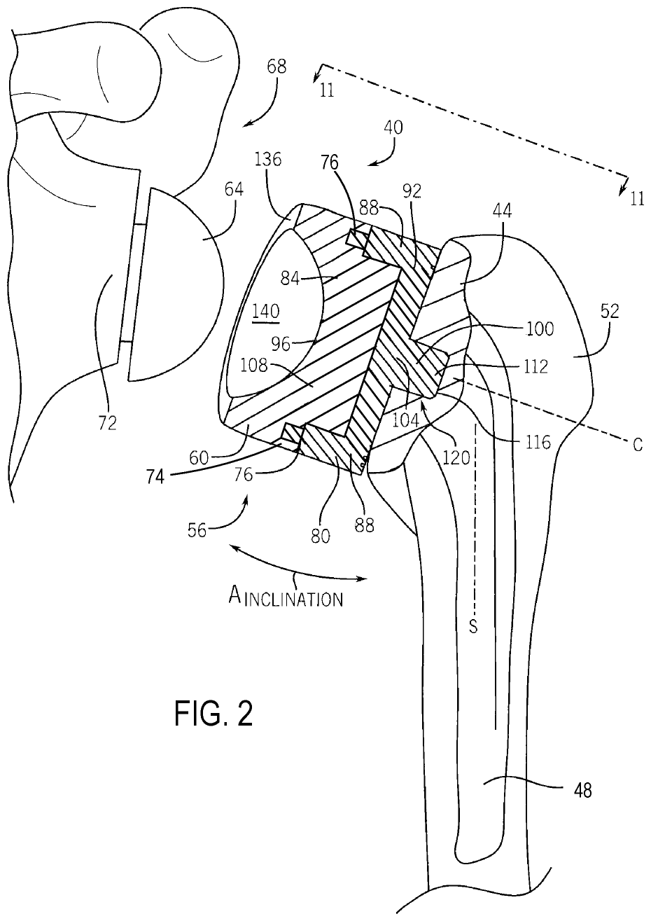

Referring to FIG. 2, a non-limiting example of a reverse shoulder prosthesis 40 with increased impingement-free range of motion as compared to FIG. 1 is shown. The humeral component 44 may include a stem 48 that extends into a bore formed within the humerus 52. The stem 48 has a longitudinal stem axis S. In some configurations, a stemless component may be used where stem 48 is omitted. A humeral tray assembly 56 has a bearing surface insert 60 that has a generally concave bearing surface 140 with a flat edge 96 and outer surface 136. The humeral tray assembly 56 may be connected to the stem 48 using a tapered shaft 116, which in a non-limiting example may form a Morse taper. The insert 60 articulates with a complementary convex hemispherical glenosphere 64 of a glenoid component 68 that is fixed within the glenoid cavity of the scapula 72. The inclination angle A may be set by the angle of a cut on the humerus 52.

In the humeral tray assembly 56, the insert 60 includes a locking tab receiver portions 74 that can receive a locking tab extension 76 of side support portion 80 of tray baseplate 88. The one or more locking tab extensions 76 may extend into the corresponding one or more receiving portions 74 of the insert 60 when being assembled with tray baseplate 88 to form tray assembly 56. The side support portion 80 may guide the insert 60 into position with the tray baseplate 88, such as by being centered on central axis C. Insert 60 includes a body 84 with an insert extension 108 configured to be received by a tray baseplate well 92. Assembling insert 60 into tray baseplate well 92 may include aligning the central axis C of the tray baseplate well 92. The humeral tray assembly 56 also includes a mounting stud 100 having a first end 104 and a second end 112 comprising a tapered shaft 116. The first end 104 of the mounting stud 100 is coupled to the tray baseplate well 92 of the tray baseplate 88. The second end 112 of the mounting stud 100 is secured in a stem opening 120 of the stem 48 by way of an interference fit or taper lock formed by impacting the mounting stud 100 in the stem opening 120. The mounting stud 100 may be impacted into stem opening 120 of the stem 48 by way of an impact tool, such as a hammer and the like.

The parts of the humeral tray assembly 56 may be formed from, for example: (i) a metal or metal alloy such as titanium, a titanium alloy (e.g., titanium-6-aluminum-4-vanadium), a cobalt alloy, a stainless steel alloy, or tantalum; (ii) a nonresorbable ceramic such as aluminum oxide or zirconia; (iii) a nonresorbable polymeric material such as polyethylene, highly cross-linked polyethylene; or (iv) a nonresorbable composite material such as a carbon fiber-reinforced polymers (e.g., polysulfone). The prosthetic component can be manufactured by machining an article formed from these materials, or by molding these materials in a suitable mold. Different materials may be used for different components of the humeral tray assembly 56. In a non-limiting example, bearing surface insert 60 is formed from polyethylene, and the tray baseplate 88 is formed from titanium.

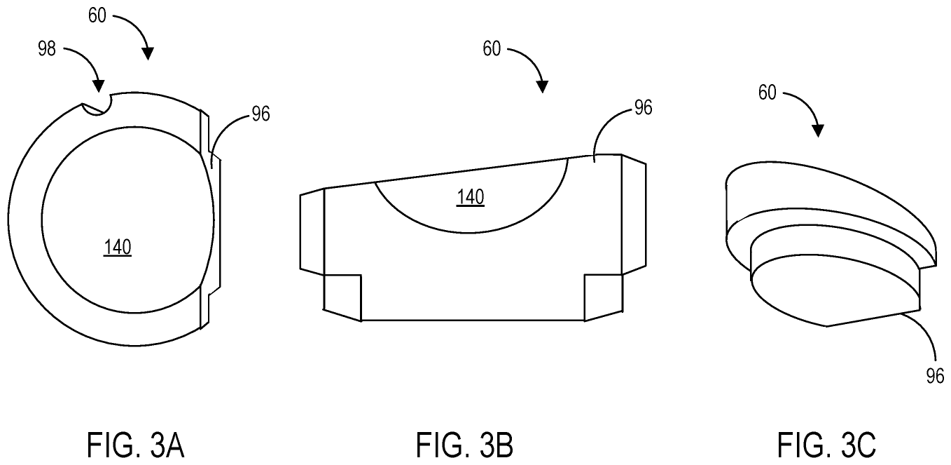

Referring to FIGS. 3A-3C, a non-limiting example of bearing surface insert 60 is shown in different views. FIG. 3A depicts a top view, FIG. 3B depicts a side view, and FIG. 3C depicts a bottom perspective view. In each view, flat surface 96 is shown that may provide for increased impingement-free range of motion by not including any implant anterior structures past the flat surface 96. This is in contrast to conventional implants that are fully round or circular where anterior structure of the implants may limit the range of motion for a subject.

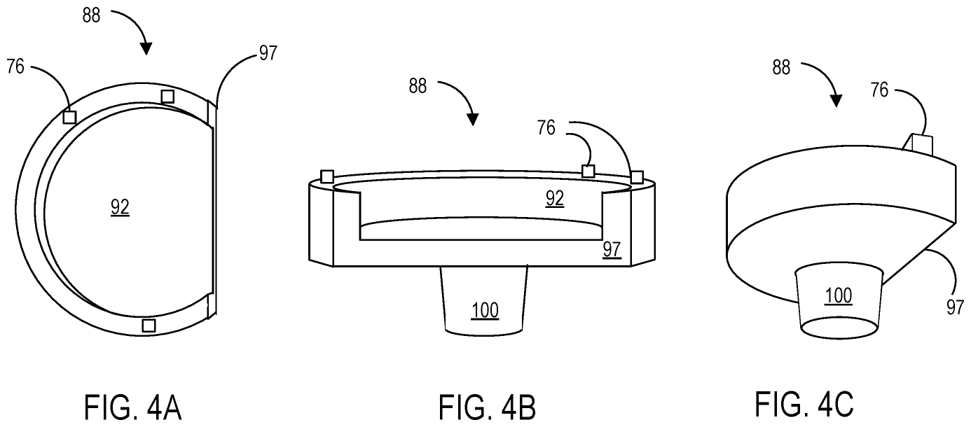

Referring to FIGS. 4A-4C, a non-limiting example of a tray baseplate 88 is shown in different views. FIG. 4A depicts a top view, FIG. 4B depicts a side view, and FIG. 4C depicts a bottom perspective view. In each view, flat surface 97 is shown that may provide for increased impingement-free range of motion by not including any implant anterior structures past the flat surface 97. Flat surface 97 of tray baseplate 88 may align with flat surface 96 of insert 60 when assembled for implantation in a subject. This is in contrast to conventional implants that are fully round or circular where anterior structure of the implants may limit the range of motion for a subject with impingement of bone near the implanted joint.

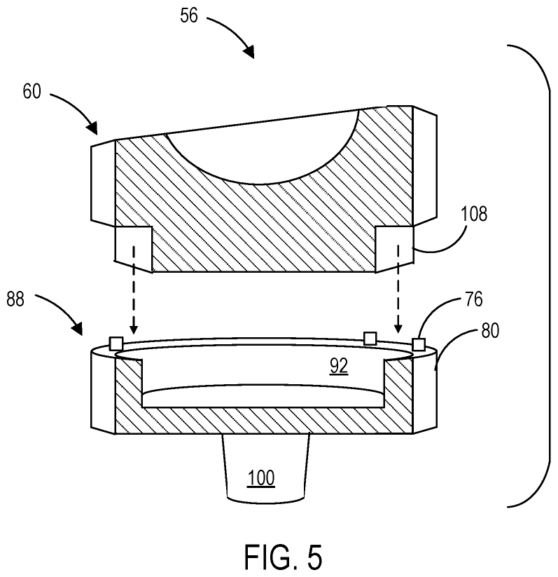

Referring to FIG. 5, an exploded perspective view is shown of a non-limiting example humeral tray assembly 56. Insert 60 may be assembled with tray baseplate 88 to form humeral tray assembly 56 for use with a reverse shoulder prosthesis 40, as shown in FIG. 1. During assembly, locking tab extensions 76 may extend into the corresponding one or more receiving portions 74 (not shown) of the insert 60. Locking tab extensions 76 may provide for alignment of the insert 60 and the tray baseplate 88 and may also provide for a locked configuration upon assembly that prevents the assembled humeral tray assembly 56 from being separated. Side support portion 80 may also guide the insert 60 into position with the tray baseplate 88. Insert 60 includes an insert extension 108 configured to be received by a tray baseplate well 92 during assembly. Flat surfaces 96 and 97 may also be used to guide a user in aligning the insert 60 and tray baseplate 88 by providing a visual reference for orienting the insert 60 and tray baseplate 88. Alignment notch 98 depicted in FIG. 3A may be used to align the insert for assembly or for implanting the assembled humeral tray assembly 56 into a subject.

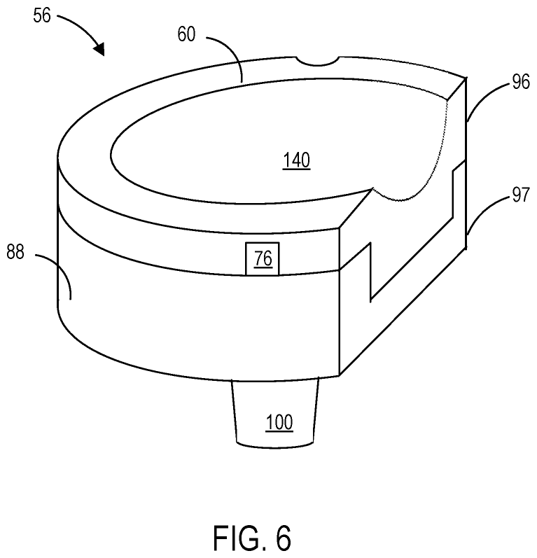

Referring to FIG. 6, a perspective view is shown of an assembled non-limiting example humeral tray assembly 56 in accordance with the present disclosure. Insert flat surface 96 is aligned with tray baseplate flat surface 97.

A method for designing the location of the insert flat surface 96 and tray baseplate flat surface 97 is also provided. A desired range of motion may be determined for a subject and the location of the insert flat surface 96 and tray baseplate flat surface 97 may be determined for a humeral tray assembly 56 based upon the desired range of motion. In a non-limiting example, larger ranges of desired motion may move the insert flat surface 96 and tray baseplate flat surface 97 towards the center of the insert 60 and tray baseplate 88. In another non-limiting example, smaller ranges of desired motion may move the insert flat surface 96 and tray baseplate flat surface 97 away from the center of the insert 60 and tray baseplate 88.

In some configurations, the desired range of motion may be determined by medical image analysis, where measurements of the maximum angle of motion may be determined to guide the location of insert flat surface 96 and tray baseplate flat surface 97. In some configurations, the desired range of motion may be determined by simulation, where 3D printed bones are generated based upon images obtained of a subject and 3D printed implants are generated and implanted into the 3D printed bones to assess a range of motion for a subject. 3D printing is a non-limiting example, and other forms of prototyping or manufacture of the simulated implants are possible, including injection molding, machining, and the like. The orientation angle of the insert flat surface 96 and tray baseplate flat surface 97 may also be adjusted in addition to the locations. The location and orientation angle of insert flat surface 96 and tray baseplate flat surface 97 may be customized to an individual patient, or population data may be used to guide the design of fixed sizes of implants for use with subjects in specified size ranges.

In some configurations, the systems and methods of the present disclosure may be used in other joints, such as the hip, knee, and the like. In some configurations, the systems and methods of the present disclosure may be used in anatomic or total arthroplasty systems, such as by providing a flat surface for the side of a head and a corresponding base plate.

The present disclosure has described one or more preferred embodiments, and it should be appreciated that many equivalents, alternatives, variations, and modifications, aside from those expressly stated, are possible and within the scope of the invention.

1. A joint prosthesis comprising:
an implant dimensioned to be implanted in a first bone of a joint of a subject;
a prosthetic insert having an insert flat surface and an outer surface dimensioned for articulation with an articular surface of an artificial joint surface of a second bone of the joint, the prosthetic insert including an extension opposite the outer surface of the insert dimensioned to be impacted into a well in a prosthetic baseplate;
the prosthetic baseplate having a baseplate flat surface configured to align with the insert flat surface, and wherein a location of the baseplate flat surface and insert flat surface is configured to provide a range of motion for the subject.

2. The joint prosthesis of claim 1, wherein the joint prosthesis is a reverse prosthesis and the location of the baseplate flat surface and insert flat surface is an anterior location when the prosthetic insert and the prosthetic baseplate are implanted in the subject.

3. The joint prosthesis of claim 1, wherein the extension is dimensioned to be impacted into the well in the prosthetic baseplate thereby forming an interference fit between the prosthetic insert and the prosthetic baseplate.

4. The joint prosthesis of claim 1, further comprising a mounting stud coupled to the prosthetic baseplate, wherein the mounting stud includes a first end and a second end, the first end being coupled to the well, the second end being dimensioned for insertion into an opening in the implant dimensioned to be implanted in the first bone thereby forming an interference fit between the prosthetic baseplate and the implant dimensioned to be implanted in the first bone.

5. The joint prosthesis of claim 4, wherein the second end of the mounting stud includes an outer surface that tapers inward from the first end to an outermost section of the second end of the mounting stud.

6. The joint prosthesis of claim 1, wherein the prosthetic baseplate includes locking tab extensions and the prosthetic insert includes provisions for receiving the locking tabs.

7. The joint prosthesis of claim 6, wherein the locking tab extensions engage with the provisions thereby forming an interference fit between the prosthetic insert and the prosthetic baseplate.

8. The joint prosthesis of claim 6, wherein the joint prosthesis includes a plurality of locking tab extensions.

9. The joint prosthesis of claim 6, wherein the locking tab extensions provide for alignment of the baseplate flat surface and insert flat surface.

10. The joint prosthesis of claim 1, wherein the first bone is a humerus and the second bone is a scapula.

11. A method for manufacturing a prosthetic component for replacing a part of a bone of a joint in a subject, the method comprising:
forming the prosthetic component to include a range of motion for the prosthetic component having been determined by
a) dimensioning an implant to be implanted in a first bone of a joint of a subject;
b) forming a prosthetic insert having an insert flat surface and an outer surface dimensioned for articulation with an articular surface of an artificial joint surface of a second bone of the joint, the prosthetic insert including an extension opposite the outer surface of the insert dimensioned to be impacted into a well in a prosthetic baseplate;
c) forming the prosthetic baseplate having a baseplate flat surface configured to align with the insert flat surface, and
d) locating the baseplate flat surface and insert flat surface to provide a range of motion for the subject.

12. The method of claim 11, wherein the prosthetic component is a reverse prosthesis and the location of the baseplate flat surface and insert flat surface is an anterior location when the prosthetic insert and the prosthetic baseplate are implanted in the subject.

13. The method of claim 11, further comprising impacting the extension into the well in the prosthetic baseplate thereby forming an interference fit between the prosthetic insert and the prosthetic baseplate.

14. The method of claim 11, further comprising coupling a mounting stud to the prosthetic baseplate, wherein the mounting stud includes a first end and a second end, the first end being coupled to the well, the second end being dimensioned for insertion into an opening in the implant dimensioned to be implanted in the first bone thereby forming an interference fit between the prosthetic baseplate and the implant dimensioned to be implanted in the first bone.

15. The method of claim 14, further comprising tapering an outer surface of the second end of the mounting stud inwardly from the first end to an outermost section of the second end of the mounting stud.

16. The method of claim 11, wherein the prosthetic baseplate includes locking tab extensions and the prosthetic insert includes provisions for receiving the looking tabs.

17. The method of claim 16, further comprising engaging the locking tab extensions with the provisions thereby forming an interference fit between the prosthetic insert and the prosthetic baseplate.

18. The method of claim 16, wherein the joint prosthesis includes a plurality of locking tab extensions.

19. The method of claim 16, further comprising aligning the locking tab extensions of the prosthetic baseplate with the provisions of the prosthetic insert to align the baseplate flat surface with the insert flat surface.

20. The method of claim 11, wherein the first bone is a humerus and the second bone is a scapula.
